(19) United States
(12) Patent Application Publication
(10) Pub. No.: US 2015/0139896 A1
(43) Pub. Date: May 21, 2015

(54) SOLAR ENERGY BASED COUNTINUOUS PROCESS AND REACTOR SYSTEM FOR THE PRODUCTION OF AN ALKENE BY DEHYDROGENATION OF THE CORRESPONDING ALKANE

(71) Applicant: SAUDI BASIC INDUSTRIES CORPORATION, Ryadh (SA)

(72) Inventors: Mohamed Sabri Abdelghani, Riyadh (SA); Mustapha Karime, Riyadh (SA); Zeeshan Nawaz, Riyadh (SA); Abdullah Mohammad Al-Qahtani, Riyadh (SA)

(21) Appl. No.: 14/396,622

(22) PCT Filed: Apr. 19, 2013
(86) PCT No.: PCT/EP2013/001161

§ 371 Date: Oct. 23, 2014

(30) Foreign Application Priority Data
Apr. 23, 2012 (EP) 12002830.3

(51) Int. Cl. C07C 5/48; C01B 3/38; C07C 5/333

(52) U.S. Cl. CPC C07C 5/48; C07C 5/3337; C01B 3/386; C07C 2529/068; C01B 2203/0277; C01B 2203/0261; C01B 2203/0855; C01B 2203/107; C01B 2203/1088
USPC 423/651; 585/658; 585/660; 422/187

(57) ABSTRACT

A solar energy based continuous process and reactor system for the production of an alkene by dehydrogenation of the corresponding alkane is performed in a reactor which process comprises alternatingly performing a first mode and a second mode in the same reactor, wherein the first mode is a non-oxidative dehydrogenation wherein the non-oxidative dehydrogenation is performed by contacting the alkane with a suitable dehydrogenation catalyst at a temperature of at least 500° C. to produce the corresponding alkene and hydrogen and wherein the second mode is an oxidative dehydrogenation wherein the oxidative dehydrogenation is performed by contacting the alkane with a suitable dehydrogenation catalyst and an oxidation agent at a temperature from 300 to 500° C. to produce the corresponding alkene wherein the dehydrogenation catalyst for the oxidative dehydrogenation and the non-oxidative dehydrogenation are the same, wherein the heat for the first mode is provided by a solar energy source.

### CROSS REFERENCE TO RELATED APPLICATIONS

This is the U.S. National Stage of Application No. PCT/EP2013/001161, filed on 19 Apr. 2013, the disclosure of which is herein incorporated by reference. Priority under 35 U.S.C. §119(a) and 35 U.S.C. §365(b) is claimed from European Application No. 12002830.3, filed 23 Apr. 2012, the disclosure of which is also incorporated herein by reference.

The invention relates to a solar energy based continuous process and reactor system for the production of an alkene by dehydrogenation of the corresponding alkane.

Such a process is described in US2010/0314294A1, which discloses a hydrocarbon dehydrogenation process in which a hydrocarbon feed, normally a straight run naphtha, comprising acyclic and cyclic paraffins is dehydrogenated at elevated temperature of at least 540° C., with process heat provided at least in part by a solar or nuclear thermal energy source.

A disadvantage of this process is that the operation of the process is dependent on the amount of available energy, which leads to fluctuations in the output. For example, in case of solar energy, the amount of energy available during night-time or cloudy weather is less than during day-time when there are no clouds and may even depend on the season.

However, the use of solar energy has many advantages as compared to the use of nuclear energy or energy coming from fossil fuels, such as advantages from an environmental, public health and safety and sustainability view point. For example, the use of solar energy may eliminate or reduce the need to generate carbon dioxide as a consequence of the burning of hydrocarbons to generate energy. For example, if solar energy is used, fossil fuel energy otherwise used for this purpose can be conserved. For example, the use of solar energy has considerably lower public health and safety concerns than nuclear energy, since unsafe operation of nuclear power plants can lead to radiation contamination of entire regions.

Therefore, it is the object of the invention to provide a process and reactor system for the production of an alkene by dehydrogenation of the corresponding alkane, in which solar energy is used as the main or sole source of energy (i.e. reaction heat) and wherein the output of the process is less dependent on the amount of available solar energy.

This object is achieved by a process for the production of an alkene by dehydrogenation of the corresponding alkane wherein the process is performed in a reactor which process comprises the steps of alternatingly performing a first mode and a second mode in the same reactor,

wherein the first mode is a non-oxidative dehydrogenation (endothermic) wherein the non-oxidative dehydrogenation is performed by contacting the alkane with a suitable dehydrogenation catalyst at a temperature of at least 500° C. to produce the corresponding alkene and hydrogen and
wherein the second mode is an oxidative dehydrogenation (exothermic)
wherein the oxidative dehydrogenation is performed by contacting the alkane with a suitable dehydrogenation catalyst and an oxidation agent at a temperature from 300 to 500° C. to produce the corresponding alkene
wherein the dehydrogenation catalyst for the oxidative dehydrogenation and the non-oxidative dehydrogenation are the same, wherein heat for the first mode is provided by a solar energy source.

The non-oxidative dehydrogenation is an endothermic process which is a process that requires heat, whereas the oxidative dehydrogenation is an exothermic process. By alternatingly performing the non-oxidative dehydrogenation and the oxidative dehydrogenation, it is possible to switch between endothermic and exothermic reactions depending on the available solar energy.

The process of the invention makes it possible to use solar energy as the main, preferably sole source of energy for the dehydrogenation of alkanes during a 24 hour (continuous) production of the same product. The process of the invention thereby combines the advantages of the use of solar energy as the main, preferably sole source of energy with less or even without fluctuations in the output (the amount of corresponding alkene produced) of the process.

Although the advantageous of solar energy are well known, so far, in the petrochemical industry, there has virtually been no mention of the use of solar energy as the main source of energy for petrochemical processes.

US2010/0314294A1 describes a hydrocarbon dehydrogenation process in which a hydrocarbon feed comprising acyclic and cyclic paraffins is dehydrogenated at elevated temperatures of at least 540° C. with process heat provided at least in part by a solar or nuclear thermal energy source.

However, such process is not suitable for 24 hour (continuous) production of the same product and the high energy required for petrochemical reactions was thought to be prohibitive for the use of solar energy as the sole source of energy.

Zhang et al., disclose the following in Chromium oxide supported on mesoporous SBA-15 as propane dehydrogenation and oxidative dehydrogenation catalysts. Catalysis Letters Vol. 83, No. 1-2, October 2002, pages 19-25: ‘Cr₂O₃/SBA-15 and Cr₂O₃/ZrO₂/SBA-15 catalysts are more selective to propene in comparison with Cr₂O₃/ZrO₂ and Cr₂O₃/γ-Al₂O₃ for non-oxidative dehydrogenation of propane. In oxidative dehydrogenation of propane by O₂ and CO₂, Cr₂O₃/SBA-15 also displays better activity, selectivity and stability than the other two supported catalysts.

However, Zhang et al do not teach that it is possible to switch between the two reactions in a continuous process.

U.S. Pat. No. 3,725,494 discloses a two-stage dehydrogenation process for producing di-olefins from mono-olefins wherein the mono-olefin stream is first dehydrogenated under non-oxidative conditions using a non-oxidative dehydrogenation catalyst comprised on potassium carbonate, iron oxide, and chromium oxide for the first phase followed by an oxidative dehydrogenation with a different catalyst, namely an iron phosphate catalyst for the second phase. Therefore, U.S. Pat. No. 372,594 teaches that different catalysts need to be used for oxidative and non-oxidative dehydrogenation, whereas the inventors use the same dehydrogenation catalyst for alternating oxidative and non-oxidative dehydrogenation of the alkane.

In the framework of the invention, with alkane is meant a hydrocarbon of formula C₂H₂ₙ₊₂. For example, the alkane can have from 2 to 10, for example from 2 to 8, preferably from 3 to 5 carbon atoms per molecule. For example, the alkane may be ethane, propane or butane, for example i-butane or n-butane, preferably propane or butane.

Alkenes formed in the invention—depending on the corresponding alkane used—include but are not limited to butene, propylene, ethylene, preferably butene or propylene.

The alkane may be used in its pure form, but may also be present in a feedstream of a mixture of alkanes or—but less suitable for large scale processes, in a feedstream of alkane (also referred to herein as alkane feedstream) with an inert gas, such as N₂. Preferably, the alkane is present in a feedstream that predominantly comprises one alkane species.

Accordingly, it is preferred that the alkane comprised in the feedstream consists of at least 35 mol % of only one alkane species, more preferably of at least 75 mol % of only one alkane species, even more preferably of at least 85 mole % of only one alkane species, particularly preferably of at least 90 mole % of only one alkane species, more particularly preferably of at least 95 mole % of only one alkane species and most preferably of at least 98 mole % of only one alkane species. This feed composition affects the product distribution.

For example, the amount of n-butane in a butane feedstream is preferably at least 96 mol % based on the total alkanes in the feedstream and for example at most 98 mol %. The other alkanes in the butane feedstream may for example be mostly i-butane, which would then be present in an amount of from 4 to for example 2 mol % based on the total alkanes in the feedstream.

For example, the amount of propane in a propane feedstream is preferably at least 94 mol % based on the total alkanes in the feedstream and for example at most 98 mol %.

A propane feedstream may for example further comprise 3-4 mol % n-butane and i-butane based on the total alkanes in the feedstream and for example 1 to 3 mol % ethane based on the total alkanes in the feedstream.

Preferably, the total amount of alkane in the feedstream is at least 98 wt %, preferably at least 99 wt %, for example at least 99.5 wt %, for example at least 99.7 wt %, for example 99.9 wt % based on the total feedstream. Small amounts of olefins (for example from 0.1 to 0.5 wt % based on the total feedstream) and trace amounts of sulphur (for example 10-100 ppm based on the total feedstream) may be present in the feedstream.

In case of the second mode (oxidative dehydrogenation), the oxidation agent is preferably present in the alkane feedstream or separately added to the reactor, so that the alkane is already partially converted to the corresponding alkene upon formation of heat, so that the alkane feedstream is heated before entering the reactor. Any known oxidation agent known in the art may be used, including, but not limited to oxygen, air, or water (steam).

With reactor is meant a device for containing and controlling a chemical reaction, in this case the oxidative and non-oxidative dehydrogenation reaction to form an alkene from the corresponding alkane.

With ‘alternatingly performing a first mode and a second mode in the same reactor’ is meant that said same reactor is operated in a cycle comprising a recurring succession of a first mode and a second mode. Said cycle may comprise as many recurrences as desired, for example said cycle may comprise at least 1 recurrence (first mode followed by second mode, followed by first mode, followed by second mode), for example at least 2, for example at least 5, for example at least 10 and/or for example at most 1000, for example at most 500, for example at most 100 recurrences and may also comprise further process steps, for example for regeneration of the dehydrogenation catalyst. The timing of the reaction mode depends largely upon the feedstock, severity of operation and type of catalyst used in the reactor system.

The first mode is a non-oxidative dehydrogenation of the alkane by contacting the alkane with a suitable dehydrogenation catalysts, examples of which are known to the skilled person and are also included herein.

The non-oxidative dehydrogenation is performed in the reactor at a temperature of at least 500° C., preferably at a temperature of 500 to 1200° C., more preferably at a temperature of 550 to 1000° C. and particularly preferably at a temperature of 550 to 750° C. and most preferably at a temperature of 550 to 650° C.

Pressure within the reactor preferably lies within a range of from 50.7 kilopascals (KPa) to 2 megapascals (MPa), more preferably from 101 KPa to 304 KPa.

During the non-oxidative dehydrogenation, the alkene is produced together with hydrogen.

Hydrogen may optionally be fed to the reactor together with the alkane, for example in case the dehydrogenation catalyst is a platinum based catalyst and a non-oxidative dehydrogenation of the alkane is conducted. In such case, the amount of alkane is preferably fed to the reactor such that the molar ratio of alkane to H₂ in the reactor in the non-oxidative dehydrogenation is in the range from 0.01-0.5.

The second mode is an oxidative dehydrogenation of the alkane by contacting the

the alkane with a suitable dehydrogenation catalyst, examples of which are known to the skilled person and are also included herein and an oxidation agent.

Examples of oxidation agents include but are not limited to O₂ or air, CO₂ and H₂O (e.g. in the form of steam).

The oxidative dehydrogenation is performed in the reactor at a temperature from 300 to 500° C., for example at a temperature from 400 to 500° C., for example at a temperature from 450 to 500° C.

The reaction pressure of the process of the present invention is not particularly critical and can vary from atmospheric to 0.5 MPa, however a reaction pressure of not more than 0.2 MPa is preferred.

In the oxidative hydrogenation, next to oxidation agent and alkane, also inert gases, such as N₂, He, Ar and the like may be present.

During the oxidative dehydrogenation, the alkene is produced together with mainly CO₂ and some CO) in case the oxidation agent is O₂ or steam. In case the oxidation agent is CO₂, during oxidative dehydrogenation, the alkene is produced together with mainly CO (and some CO₂).

In the invention, the dehydrogenation catalyst is the same for both the oxidative and the non-oxidative dehydrogenation. Examples of dehydrogenation catalysts include but are not limited to dehydrogenation catalyst systems comprising a catalyst, optionally a support and optionally a promoter. Examples of catalysts include platinum and chromium-based catalysts with various promoters, with for example acidic/non-acid supports, such as for example described in U.S. Pat. No. 5,132,484, U.S. Pat. No. 3,488,402, U.S. Pat. No. 2,374,404, U.S. Pat. No. 3,679,773, U.S. Pat. No. 4,000,210, U.S. Pat. No. 4,177,218, CN200910091226.6; Pak Pat. 140812, JP61238345, JP04349938 and WO/2005/040075, hereby incorporated by reference.

Preferably, there is a promoter present in the dehydrogenation catalyst used in the invention. For example, such promoter may be selected from Groups III, IVA, VIB or VIII of the Periodic Table, for example as disclosed in U.S. Pat. No. 2,814,599 and U.S. Pat. No. 3,679,773-A, hereby incorporated by reference.

Also mixtures of promoters may be used. For example alkali and alkaline earth metals, for example Na, Ca, K, etc. may be used as secondary promoters to neutralize the acidity of support as declared in U.S. Pat. No. 5,146,034 and U.S. Pat. No. 3,899,544.

As a support, any support may be used, for example an alumina or zeolite support may be present in the dehydrogenation catalyst used in the invention.

Preferably, a zeolite support is used, for example ZSM-5 and SAPO-34 zeolite support may be used as disclosed in U.S. Pat. No. 5,416,052, U.S. Pat. No. 5,146,034, U.S. Pat. No. 0,110,630A1, U.S. Pat. No. 3,442,794, U.S. Pat. No. 4,489,216, CN200910091226.6, CN201010103170.4 and PK140812, hereby incorporated by reference.

Advantages of the use of a zeolite support is that they may limit corrosion problems, may lead to a high yield and/or it may reduce coke formation due to a larger surface area.

Preferably, the dehydrogenation catalyst is a platinum or chromium based catalyst, preferably further comprising a promoter and/or a support, for example a zeolite or amorphous alumina support, preferably on a zeolite support.

As used herein, the term “zeolite” relates to an aluminosilicate molecular sieve. These inorganic porous materials are well known to the skilled person. An overview of their characteristics is for example provided by the chapter on Molecular Sieves in Kirk-Othmer Encyclopedia of Chemical Technology, Volume 16, p 811-853; in Atlas of Zeolite Framework Types, 5^{th} edition, (Elsevier, 2001).

The optimal amount of catalyst can be determined by the skilled person through routine experimentation, for example the weight hourly space velocity, that is the ratio of the weight of the alkane which comes in contact with a given weight of catalyst per unit time may be chosen in the range of 1 to 10 h⁻¹, for example in the range of 4 to 8 h⁻¹.

The heat for the first mode (the heat provided to the reactor) is provided by a (non-fossil) solar energy source.

The term “solar energy source” is used herein with its generally accepted meaning, which means that any fossil sources of energy are excluded (i.e. the energy source is non-fossil). Accordingly, the term “solar energy source” is meant to reflect any non-fossil source of energy that is at least partially, preferably entirely, provided by radiant light and/or heat energy from the sun. In one embodiment, accordingly, the present application provides a process wherein heat for the “first mode” as defined herein is provided by radiant light and/or heat energy from the sun. This is in contrast with a conventional process for non-oxidative dehydrogenation of an alkane wherein the process heat is exclusively provided by non-renewable sources such as by burning a fossil fuel source.

The solar energy source may use any technology to capture the energy from the sun, for example the thermal energy of the sun. In the case of thermal energy, light is converted into heat energy. This is typically achieved by focussing solar radiation onto a point source using mirrors, causing the point source to increase in temperature thereby generating heat. For commercial applications, usually multiple mirrors are used to increase the capture of light and the mirrors may be moved during the day to change the optimal position of the mirrors (and follow the sun) during the day.

Alternatively, the sun's heat may be directly absorbed by a heat transfer fluid or solid particles.

The solar energy may be used to heat water, which can then generate steam, which in its turn can power turbines for generating electricity, which may then be used to drive the dehydrogenation or the steam may be used for directly heating the reactor. The water may be heated directly by solar irradiation or by a heat transfer fluid, for example a molten salt or a thermal oil that is heated by solar irradiation. The temperature of the steam in these technologies can be saturated or superheated steam that can reach 500° C. in parabolic troughs and linear Fresnel technologies and may reach even higher temperature of around 540° C. in case of power tower with steam pressure reaching to 100 bars as for example described in U.S. Pat. No. 7,296,410.

In another type of solar power towers, solid particles can be heated to a temperature reaching 850 to 1000° C. (as for example described in U.S. Pat. No. 4,777,934) by direct absorption of solar heat through an open window in the tower top. In this so-called particle received tower, particles such as sand, are used as heat transfer media with air and can therefore generate air temperatures of higher than 700° C.

Preferably, in the invention, the solar energy source is selected from the group consisting of a solar power tower, for example a solar power tower using steam or solid particles, such as sand, to absorb the solar energy and a reflector-type heating system which, for example uses a heat transfer fluid to absorb the solar energy. For example, the solar energy source is a particle solar power tower that is a solar power tower using solid particles to absorb the solar energy. As used herein, the term “solar energy unit” relates to a unit comprising the solar energy source.

Examples of (particle) solar power towers are known to the person skilled in the art, for example as described in Proceeding of 2010 SOLARPACES Conference on concentrated solar power, Perpignan, 2010, the solar power tower produces hot air using a particle tower received where hot sand is heated by the sun to temperature of for example 850 to 1000° C. and air is used to exchange heat with the sand, giving the air an air temperature of above 650° C. This air can be used in the process of the invention to provide heat in the first mode (operation during day-time when there is sufficient sun).

The hot particles, such as sand in a particle solar power tower may also be used to store heat in a particle (e.g. sand) bed for providing heat (for example in the form of heated air, which will have a lower temperature than during day-time to the process of the invention in the second mode (operation during night-time or when there is insufficient sun).

The heat (e.g. in the form of steam or solid particles) from the solar energy source may be transferred to the reactor directly, or indirectly by using a heat exchanger that generates hot air or hot heat transfer fluid or solid particles which can be used to heat the reactor. Such a reactor is also known as a solar catalytic reactor.

In the exothermic oxidative dehydrogenation, heat for the second mode is provided by oxidation of the alkane, which oxidation preferably already partially occurs before the alkane enters the reactor. Preferably, in the second mode, the heat from the alkene that is produced is preferably transferred to the alkane to (further) heat the alkane before it enters the reactor, for example by using a heat exchanger.

The heat for the first mode may further be provided by the alkene produced in the first mode, for example by using a second heat exchanger in which the heat from the alkene produced in the first mode is used to heat the reactor or to heat alkane that is fed to the reactor.

Heat for the second mode may be further provided by the solar energy source. For example by using a second heat exchanger in which the heat from the solar energy source, for example heated sand that contains solar energy that was generated during the day is transferred to the reactor or to heat alkane that is fed to the reactor.

In the first mode, the non-oxidative dehydrogenation of the alkane, hydrogen is formed as a byproduct. In the second mode, the oxidative dehydrogenation, carbon dioxide is formed as a byproduct.

Therefore, the process of the invention may further comprise using the hydrogen formed in the process in other chemical processes using hydrogen as a feed component, for instance for hydrogenation purposes in for example petrochemical plants.

The dehydrogenation catalyst may be regenerated, for instance by recycling and therefore, the process of the invention may further comprise the step of regenerating or recycling of the dehydrogenation catalyst. Regenerating or recycling of the dehydrogenation catalyst may be performed using methods known to the skilled person, for instance by burning of coke deposits in an oxygen containing atmosphere.

For example, a dehydrogenation catalyst based on Pt may be regenerated by subsequently using

(i) steam, for example hot air (of for example 550 to 650° C.) which may for example be generated by the solar energy source, for example a solar power tower
(ii) redispersion of the catalyst using chlorination, for example at a temperature from 450 to 550° C.
(iii) dechlorination after redispersion of the catalyst, for example using steam of a temperature of from 450 to 550° C., for example obtained as a result of the steam step of step (i) and
(iv) reduction with H₂ at 475 to 525° C.

For example, a dehydrogenation catalyst based on Cr may be regenerated by subsequently using

(i) steam, for example at a temperature of 350 to 450° C.
(ii) hot air, which may for example be generated by the solar energy source, for example a solar power tower, for example at a temperature of 550 to 650° C. and
(iii) evacuation
(iii) reduction with H₂ at 475 to 525° C.

In another aspect, the invention relates to an alkene obtainable by the process of the invention].

An alkene obtainable by the process of the invention is produced using less fossil or nuclear energy sources than the conventionally produced alkene, since solar energy is used as its energy source.

In another aspect, the invention also relates to a reaction system suitable for performing the process of the invention in. This reaction system is presented in FIG. 1 (FIG. 1) and FIG. 2 (FIG. 2).

FIG. 1 schematically represents the operation of a reaction system comprising a reactor (1), a first heat exchanger (2), a solar energy source (3) and optionally a second heat exchanger (4), optionally a separation unit (5) during the first mode (non-oxidative dehydrogenation).

FIG. 2 schematically represent the operation of a reaction system comprising a reactor (1), a first heat exchanger (2), a solar energy source (3) and optionally a second heat exchanger (4), optionally a separation unit (5) during the second mode (oxidative dehydrogenation).

In particular, the invention relates to a reaction system suitable for the production of an alkene by dehydrogenation of the corresponding alkane comprising: a novel solar reactor (1), a first heat exchanger (2) and a solar energy unit (3)

wherein the novel solar reactor (1) comprises

a first inlet for receiving a heated alkane (20)

a first outlet for providing a heated alkene (30)

a second inlet for receiving an oxidation agent (100) to the reactor (1) and

a dehydrogenation catalyst

wherein the first heat exchanger (2) comprises

a first inlet for receiving an optionally preheated alkane (10)

a first outlet connected to the inlet of the reactor (1) for providing the heated alkane (20) to the reactor (1)

a second inlet for receiving heat (40) from the solar energy unit (3) and

a third inlet for receiving heat (80) from the heated alkene (30) provided by the first outlet of the reactor (1)

a second outlet for providing cooled alkene (110)

wherein the solar energy unit (3) comprises a first outlet for providing heat (40) to the first heat exchanger (2), which first outlet is connected to the second inlet of the first heat exchanger (2)
wherein the reaction system comprises a switch which allows to change between a first mode and a second mode, wherein in the first and second mode
the reactor (1) receives the heated alkane (20) via the first inlet
the reactor (1) provides the heated alkene (30) via the first outlet
the first heat exchanger (2) receives the optionally preheated alkane (10) via the first inlet
the first heat exchanger (2) provides the heated alkane (20) to the reactor (1) via the first outlet
and wherein in the first mode
the first heat exchanger (2) receives heat (40) from the solar energy unit (3) via the second inlet
   and wherein in a second mode
   the first heat exchanger (2) receives heat (80) from the heated alkene (30) provided by the first outlet of the reactor (1) via the third inlet

the first heat exchanger (2) provides cooled alkene (110) and

the reactor (1) receives the oxidation agent (100) via the second inlet.

The ‘switch’ includes any means for allowing the switch between the first and second mode of operation of the reaction system, for example one or more valves.

It should be appreciated that the second inlet of the reactor (1) for the oxidation agent (100) may be combined with the first inlet of the reactor (1) for receiving the heated alkane and that the oxidation agent may already be present in the heated alkane feed (20), the optionally preheated alkane feed (10) or even in the cold alkane feed (60). The presence of the oxidation agent (100) will cause the alkane to be converted to the corresponding alkene already in the alkane feed, under formation of CO, CO₂ and heat, which means that the alkene feed is heated before entering the reactor.

Preferably, the invention relates to a reaction system

wherein the reactor (1) further comprises a third inlet for receiving heat (130) from the first heat exchanger (2)
and wherein in the first and/or second mode, the reactor (1) receives heat (130) from the first heat exchanger (2) via the third inlet.

Preferably, the invention relates to a reaction system,

wherein the reactor (1) further comprises a second outlet for providing heat (50) to the solar energy unit (3) and

wherein the solar energy unit (3) further comprises a first inlet for receiving heat (50), which first inlet is connected to the second outlet of the reactor (1)

and wherein in the first and/or second mode, the solar energy unit (3) is provided with heat (50) from the reactor (1) via the first inlet.

Preferably, the invention relates to a reaction system, further comprising a second heat exchanger (4) which second heat exchanger (4) comprises

a first inlet for receiving heat (70) from the heated alkene (30) provided by the first outlet of the reactor (1)

a second inlet for receiving an alkane (60)

a third inlet for receiving heat (90) from the solar energy unit (3)
a first outlet for providing a cooled alkene (120)
a second outlet for providing preheated alkane (10) connected to the first inlet of the first heat exchanger (2)

wherein the solar energy unit (3) further comprises a second outlet for providing heat (90) to the second heat exchanger (4), which second outlet is connected to the third inlet of the second heat exchanger (4)

wherein in the first and second mode
the second heat exchanger (4) receives an alkane (60) via the second inlet

the second heat exchanger (4) provides preheated alkane (10) to the first heat exchanger (2) via the second outlet

preferably, wherein in the first mode,
the second heat exchanger (4) receives the heat (70) from the heated alkene (30) provided by the first outlet of the reactor (1) via the first inlet and
the second heat exchanger (4) provides cooled alkene (120) via the first outlet.
preferably, wherein in the second mode,
the second heat exchanger (4) receives the heat (90) from the solar energy unit (3) via the third inlet
   preferably,
wherein reactor (1) further comprises an inlet for receiving heat (130) from the first heat exchanger (2) and

wherein in the first mode, the reactor (1) receives heat (130) from the first heat exchanger (2).

preferably,
wherein reactor (1) further comprises an inlet for receiving cold (140) from the second heat exchanger (4) and

wherein in the second mode, the reactor (1) receives cold (140) from the second heat exchanger (4).

FIG. 3 (FIG. 3) schematically represents a reactor (1) that is suitable for use in the process and reaction system of the invention.

The reactor (1) represented in FIG. 3 is advantageously used in the process and reaction system of the invention, since it is capable of controlling the exothermic heat produced in the second mode by dissipating its heat into the hot air on the shell side in such a way as to maintain an isothermal reactor performance. During non-oxidative dehydrogenation (first mode), the presence of the shell is also advantageous as it provides the possibility to supply heat (from the solar energy source) to the catalyst bed so that also during the first mode an isothermal reactor performance is maintained.

In particular, FIG. 3 represents a reactor (1) that is suitable for use in the process and reaction system of the invention,

wherein the reactor comprises a shell (200) and tubes (210)

wherein the tubes comprise the dehydrogenation catalyst, the first inlet for receiving a heated alkane (20) and the first outlet for providing the heated alkene (30) and the second inlet for receiving an oxidation agent (100) and

wherein the shell comprises an inlet for receiving heat (130) from the first heat exchanger (2) and optionally the fourth inlet for receiving cold (140) from the second heat exchanger (4),

wherein the shell comprises an outlet for heat (220) from the reactor (1).

Preferably, the invention relates to a reaction system of the invention,

wherein the reactor (1) further comprises a separation unit (5) which comprises

an inlet for receiving the cooled alkene (110; 120) and a first outlet for alkene (170) and a second outlet for the remaining products comprised in the dehydrogenation reaction product (180).

Accordingly, the present invention provides reaction system according to any one of the herein described embodiments

wherein the reactor (1) comprises a shell (200) and tubes (210)

wherein the tubes comprise the dehydrogenation catalyst, the first inlet for receiving a heated alkane (20) and the first outlet for providing the heated alkene (30) and the second inlet for receiving an oxidation agent (100) and

wherein the shell comprises an inlet for receiving heat (130) from the first heat exchanger (2) and optionally the fourth inlet for receiving cold (140) from the second heat exchanger (4),

wherein the shell comprises an outlet for heat (220) from the reactor (1).

Furthermore, the present invention provides reaction system according to any one of the herein described embodiments,

wherein the reactor (1) further comprises a separation unit (5) which comprises

an inlet for receiving the cooled alkene (110; 120) and a first outlet for alkene (170) and a second outlet for the remaining products comprised in the dehydrogenation reaction product (180).

Preferably, the invention relates to a reaction system, further comprising means for regeneration of the dehydrogenation catalyst.

In another aspect, the invention relates to the use of the reaction system of the invention for producing an alkene.

Although the invention has been described in detail for purposes of illustration, it is understood that such detail is solely for that purpose and variations can be made therein by those skilled in the art without departing from the spirit and scope of the invention as defined in the claims.

It is further noted that the invention relates to all possible combinations of features described herein, preferred in particular are those combinations of features that are present in the claims.

It is further noted that the term ‘comprising’ does not exclude the presence of other elements. However, it is also to be understood that a description on a product comprising certain components also discloses a product consisting of these components. Similarly, it is also to be understood that a description on a process comprising certain steps also discloses a process consisting of these steps.

### BRIEF DESCRIPTION OF THE FIGURES

FIG. 1 schematically represents the operation of a reaction system comprising a reactor (1), a first heat exchanger (2), a solar energy source (3) and optionally a second heat exchanger (4), optionally a separation unit (5) during the first mode (non-oxidative dehydrogenation).

FIG. 2 schematically represent the operation of a reaction system comprising a reactor (1), a first heat exchanger (2), a solar energy source (3) and optionally a second heat exchanger (4), optionally a separation unit (5) during the second mode (oxidative dehydrogenation).

FIG. 3 (FIG. 3) schematically represents a reactor (1) that is suitable for use in the process and reaction system of the invention.

FIG. 4 (FIG. 4) schematically shows the regeneration steps of the dehydrogenation catalyst.

The invention will now be elucidated by way of the following examples, without however being limited thereto.

### EXAMPLES

### Example 1

### Propane Dehydrogenation

A Pt-based and zeolite supported catalyst (catalyst A) was prepared and treated in accordance with CN200910091226.6 and PK140812. Weight hourly space velocity (WHSV) is the ratio of the weight of the alkane which comes in contact with a given weight of catalyst per unit time. Measured amount of the catalyst was thereafter employed in a micro-reactor to achieve desired WHSV (6 h⁻¹) and the molar ratio of hydrogen to propane feedstock was 0.24. Reaction temperature in the reactor was 590° C. and 99.2% pure propane was used. At first, the catalysts were dechlorinated/regenerated at 500° C. with N₂ mixed steam, where the steam flow rate is 0.05-2 ml/g of catalyst/hr. Then reduced under flowing H₂ (1-15 ml/g/min) at 500° C.

The reaction mixture composed of H₂ and C₃H₈ in different molar ratios (0.01-0.5) whatever feasible for reaction equilibrium of non-oxidative dehydrogenation (PDH). Similarly, for oxidative dehydrogenation (ODH) the reaction mixture is composed of C₃H₈, Air/O₂ and/or H₂O or inert gases like N₂, He, Ar, etc.

The chlorination process is carried out to redisperse Pt (re-activate catalyst) in similar setups. The catalyst is heated to 500° C. in a flow of N₂, with temperature rising rate 5° C./min. The C₂Cl₂H₄ solution flask is immersed in the water bath of 0° C. (flow rate 2-20 ml/g/hr to the reactor for 0.25-2 hr), and exhaust gases are passed through concentrated NaOH solution. The dispersed samples were again dechlorinated and then reduced before reaction tests in particular PDH.

The analysis results are shown in Table 1 in wt %. After this 5.45 h or 9 h operation depending upon process the catalyst bed was regenerated and ODH was started respectively. The results of ODH at 490° C. are shown in Table 2. TOS stands for time-on-stream, which is the time during which the alkane (feedstream) is fed to the reactor.

TABLE 1
Catalyst A for Propane dehydrogenation - PDH (non-
oxidative dehydrogenation or ‘first mode’)

| 590° C., 1 atm, | | | |
|---|---|---|---|
| WHSV = 6 h⁻¹ | TOS = 15 min | TOS = 5.45 h | TOS = 9 h |
| Propylene Selectivity (%) | 87.15 | 88.32 | 92.51 |
| Total Olefin Selectivity (%) | 91.56 | 91.88 | 96.19 |
| Propane Conversion (%) | 24.57 | 21.96 | 12.45 |
| Propylene Yield (%) | 21.42 | 19.4 | 11.52 |

TABLE 2
Catalyst A for Propane dehydrogenation at 490° C. - ODH
(oxidative dehydrogenation of ‘second mode’)

| | | | CO | C₃H₆ |
|---|---|---|---|---|
| Propane/air | Propane | CO₂ Selectivity | Selectivity | Selectivity |
| ratio | Conversion (%) | (%) | (%) | (%) |
| 2:1 | 56.13 | 26.29 | 13.1 | 60.2 |

Table 1 was taken as a guideline for modelling of this reaction and in simulating the concept of using solar reactor using one of the commercially available software in the market. A solar heating of air was considered using a solid particle receiver tower which can heat sand up to 1000° C. and exchange its heat with air to about 700° C.

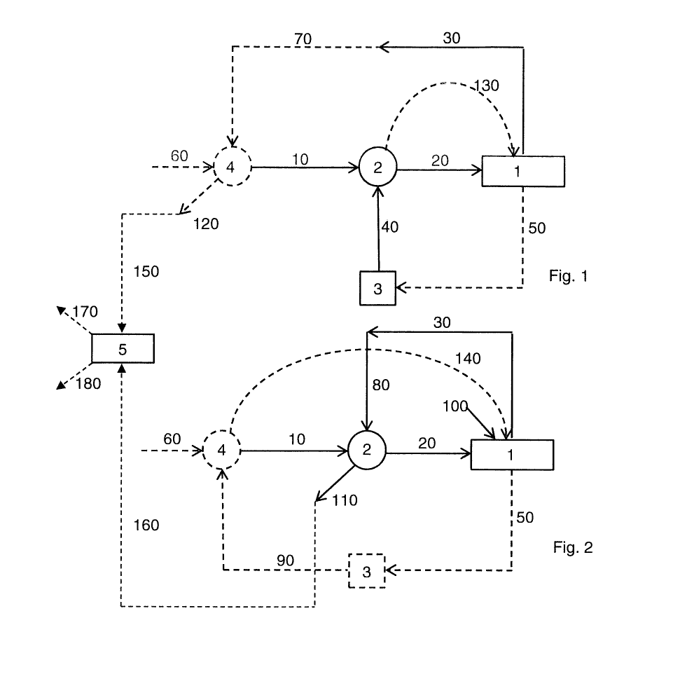

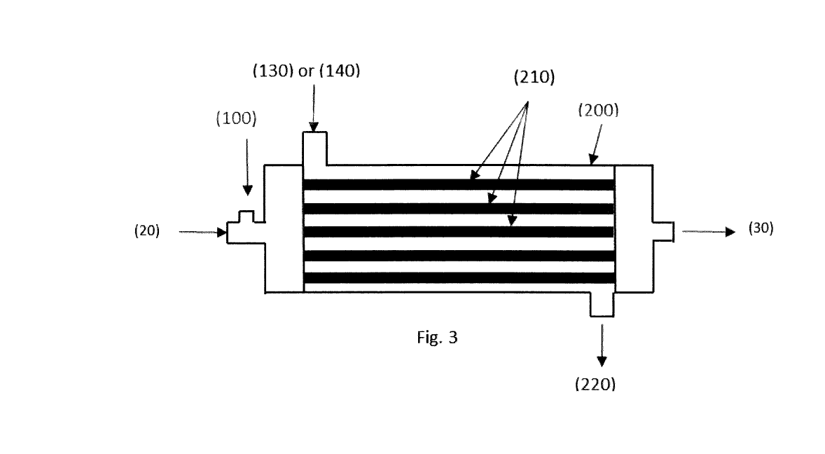

A conceptual design was made using modelling. This conceptual design is a special embodiment of the invention, which is described hereinafter, using the more general descriptors of FIG. 1 and FIG. 2.

In the conceptual design, during daytime when the sun can provide energy to the solar power tower (3), the feed propane (60) of around 30° C. is preheated against the outlet propylene product (30, 70) in the second heat exchanger (4), to produce a preheated propane (10) of around 370° C., followed by heating against outlet hot air (40) from the solar power tower (3) in the first heat exchanger (2) so that the inlet temperature of the heated alkane (20) has a temperature of 590° C. at the reactor (1) inlet. The feed enters the catalytic bed comprised in the reactor where its temperature is maintained in the dehydrogenation range by supplying heat to the bed shell side (200) of the reactor tubes using the outlet solar air (130, 140) from the first heat exchanger (2) at about 650° C. The product propylene (30) exits the reactor at 570° C. and is fed (70) to second heat exchanger (4) and the hot air (50) exits the reactor (1) at about 595° C. and is recycled back to solar power tower (3). For a reaction system using a Pt based catalyst, it is calculated that, for optimal results, a particle solar tower with an energy of 0.4 MW/ton propane/h would be required with a sand temperature of 850° C. to 1000° C. A hot air (40) of about 700° C. was found sufficient for regeneration of a Pt based catalyst.

Propane dehydrogenation was also performed with a commercially available chromium based catalyst. At inlet temperature of 590° C., conversion of 45% and selectivity of 85% was achieved. For optimal results, a particle solar tower with energy of 0.6 MW/ton propane/hr is required. During regeneration cycle, hot air (40) at 650° C. already generated by the particle receiver tower can be used directly to regenerate the catalyst and also to produce steam at 350-450° C. which is enough for steam purge.

At night time, when the sun no longer provides energy to the solar power tower (3), the reactor catalyst bed is run with a feed temperature of 490° C. in the second mode, oxidative dehydrogenation producing propylene as shown in Table 2. The reaction is exothermic and the exit reactor temperature is 550-600° C. which can be used for final preheating of the alkane feed (10) from 420° C. to 490° C. in a first heat exchanger (2). The feed can be initially heated in a second heat exchanger (4) against hot air (90) produced from the stored heated bed by solar energy as in FIG. 2. For optimal production, a minimum temperature of about 420° C. for the alkane feed (10) from the second heat exchanger (4) was found to be sufficient which requires on average a hot air (90) temperature of about 525° C. to preheat the alkane feed (60) of around 30° C. This temperature is about 175° C. lower than during the day and this suits the feature of the lower air temperature produced by the stored energy in the hot bed at night time.

Cold (140) from the air from the second heat exchanger (4) at a temperature of 445° C. is optionally used to prevent the reactor from overheating by maintaining the temperature of the reactor at the desired value of 490° C. Hot air (50) exits the reactor (1) at about 445° C. and is recycled back to solar power tower (3).

### Example 2

### Butane Dehydrogenation

The catalyst A from example 1 was also analysed under identical conditions for butane dehydrogenation. The experiment started with non-oxidative then switched to oxidative dehydrogenation. The results are given in Table 3 and 4.

TABLE 3
Catalyst A for Butane dehydrogenation - Non-oxidative

| 585° C., 1 atm, | | | |
|---|---|---|---|
| WHSV = 6 h⁻¹ | TOS = 15 min | TOS = 5.45 h | TOS = 9 h |
| Total Olefin Selectivity (%) | 74.25 | 67.08 | 66.23 |
| Butane Conversion (%) | 78.17 | 64.21 | 64.11 |

TABLE 4
Catalyst A for Butane dehydrogenation at 490° C. - ODH

| | | | | *Olefins |
|---|---|---|---|---|
| Butane/air | Butane | CO₂ Selectivity | CO Selectivity | Selectivity |
| ratio | Conversion (%) | (%) | (%) | (%) |
| 2:1 | 61.19 | 18.56 | 10.2 | 71.02 |

*Olefins Selectivity includes butane and propylene

Similar to example 1, a simulation model was set up and the above tables were used to simulate the operation of a reaction system of the invention for butane dehydrogenation. It was found that, for optimal production, for a solar particle tower a power of 0.56 MW/ton butane/h is required with a sand temperature from 850° C. to 1000° C.

At night the dehydrogenation may be switched to the second mode, the oxidative dedrogenation results of which are given in table 4 when no solar heating is available. The process can be sustained as described above.

### Example 3

### Regeneration of the Dehydrogenation Catalyst

The performance of prepared Pt based zeolite and/or alumina oxide supported catalyst samples was also investigated. While, the stable conversion/activity range is about 5-7 hr (optimum 5.45 hr is chosen) which includes PDH-Regeneration-ODH-Regeneration. The catalyst regeneration can be performed by replacing the propane flow with a flow of oxygen or steam for 1 hr followed by 15 min chlorination then 30 min dechlorination by steam or oxygen and about 1.75 hr reduction, respectively. These timings can also be manipulated by injection rate. Therefore, in total the non-oxidative dehydrogenation cycle time is about 8.7 hr. On the other hand the oxidative dehydrogenation cycle is about 15.3 hr.

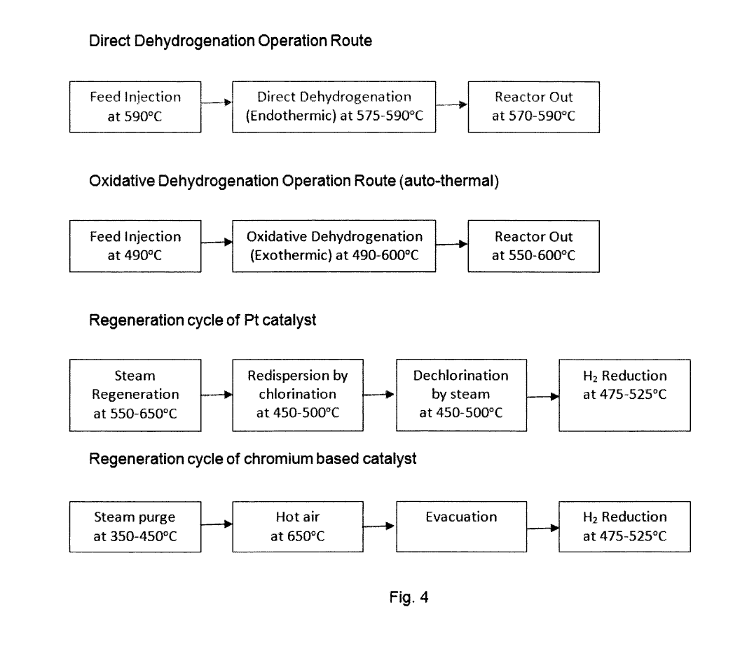

For switching non-oxidative dehydrogenation (PDH) to oxidative dehydrogenation (ODH), it depends on the choice either to regenerate or to skip regeneration depending upon the performance of catalyst and process suitability. The regeneration steps of a Pt based catalyst are shown in FIG. 4 (FIG. 4).

For a chromium based catalyst no chlorination is required. In commercial application the propane dehydrogenation is conducted for 9-12 minutes with an inlet temperature of 590° C. followed by steam purge then hot air at 650° C. followed by evacuation then reduction. The overall cycle time is 23 minutes then the cycle is repeated as shown in FIG. 4.

As has been found by calculations, for both types of catalyst (Pt and Cr based), the dehydrogenation and regeneration heat requirements can be met by the heat generated by a particle solar power tower, since hot air temperatures produced by said tower are at least 670° C. as described above.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A process for the production of an alkene by dehydrogenation of the corresponding alkane wherein the process is performed in a reactor
which process comprises alternatingly performing a first mode and a second mode in the same reactor,
wherein said first mode is a non-oxidative dehydrogenation wherein the non-oxidative dehydrogenation is performed by contacting the alkane with a suitable dehydrogenation catalyst at a temperature of at least 500° C. to produce the corresponding alkene and hydrogen and
wherein said second mode is an oxidative dehydrogenation
wherein the oxidative dehydrogenation is performed by contacting the alkane with a suitable dehydrogenation catalyst and an oxidation agent at a temperature from 300 to 500° C. to produce the corresponding alkene
wherein the dehydrogenation catalyst for the oxidative dehydrogenation and the non-oxidative dehydrogenation are the same, and wherein heat for the first mode is provided by a solar energy source.

2. The process of claim 1, wherein the heat for the first mode is further provided by the alkene produced in the first mode.

3. The process of claim 1, wherein heat for the second mode is further provided by the solar energy source.

4. The process of claim 1, wherein hydrogen produced in the process is used in other chemical processes using hydrogen as a feed component.

5. The process of claim 1, further comprising regenerating the dehydrogenation catalyst.

6. The process of claim 1, wherein the solar energy unit is selected from the group consisting of a particle solar power tower and a reflector-type heating system.

7. The process of claim 1, wherein the alkane is propane or butane.

8. The process of claim 1, wherein the dehydrogenation catalyst is platinum or chromium based catalyst.

9. A reaction system suitable for the production of an alkene by dehydrogenation of the corresponding alkane comprising:
a reactor, a first heat exchanger and a solar energy unit
wherein the reactor comprises
a first inlet for receiving a heated alkane
a first outlet for providing a heated alkene
a second inlet for receiving an oxidation agent and
a dehydrogenation catalyst
wherein the first heat exchanger comprises
a first inlet for receiving an optionally preheated alkane
a first outlet connected to the inlet of the reactor for providing the heated alkane to the reactor
a second inlet for receiving heat from the solar energy unit and
a third inlet for receiving heat from the heated alkene provided by the first outlet of the reactor
a second outlet for providing cooled alkene
wherein the solar energy unit comprises a first outlet for providing heat to the first heat exchanger, which first outlet is connected to the second inlet of the first heat exchanger
wherein the reaction system comprises a switch which allows to change between a first mode and a second mode, wherein in the first and second mode
the reactor receives the heated alkane via the first inlet
the reactor provides the heated alkene via the first outlet
the first heat exchanger receives the optionally preheated alkane via the first inlet
the first heat exchanger provides the heated alkane to the reactor via the first outlet
and wherein in the first mode
the first heat exchanger receives heat from the solar energy unit via the second inlet
and wherein in a second mode
the first heat exchanger receives heat from the heated alkene provided by the first outlet of the reactor via the third inlet
the first heat exchanger provides cooled alkene and
the reactor receives the oxidation agent via the second inlet.

10. The reaction system of claim 9,
wherein the reactor further comprises a third inlet for receiving heat from the first heat exchanger and
wherein in the first and/or second mode, the reactor receives heat from the first heat exchanger via the third inlet.

11. The reaction system of claim 9,
wherein the reactor further comprises a second outlet for providing heat to the solar energy unit and
wherein the solar energy unit further comprises a first inlet for receiving heat, which first inlet is connected to the second outlet of the reactor
and wherein in the first and/or second mode, the solar energy unit is provided with heat from the reactor via the first inlet.

12. The reaction system of claim 9, further comprising a second heat exchanger which second heat exchanger comprises
a first inlet for receiving heat from the heated alkene provided by the first outlet of the reactor
a second inlet for receiving an alkane
a third inlet for receiving heat from the solar energy unit
a first outlet for providing a cooled alkene
a second outlet for providing preheated alkane connected to the first inlet of the first heat exchanger
wherein the solar energy unit further comprises a second outlet for providing heat to the second heat exchanger, which second outlet is connected to the third inlet of the second heat exchanger
wherein in the first and second mode
the second heat exchanger receives an alkane via the second inlet
the second heat exchanger provides preheated alkane to the first heat exchanger via the second outlet

13. The reaction system of claim 12, wherein in the first mode,
the second heat exchanger receives the heat from the heated alkene provided by the first outlet of the reactor via the first inlet and
the second heat exchanger provides cooled alkene via the first outlet.

14. The reaction system of claim 12, wherein in the second mode,
the second heat exchanger receives the heat from the solar energy unit via the third inlet.

15. (canceled)

16. The process of claim 8, wherein the dehydrogenation catalyst further comprises a promoter and/or a support.
